(19) United States
(12) Patent Application Publication
(10) Pub. No.: US 2016/0296205 A1
(43) Pub. Date: Oct. 13, 2016

(54) Medical Device Anchor

(71) Applicants: Gregory Hays, Onsted, MI (US); Julia Hays, Onsted, MI (US)

(72) Inventors: Gregory Hays, Onsted, MI (US); Julia Hays, Onsted, MI (US)

(21) Appl. No.: 14/683,576

(22) Filed: Apr. 10, 2015

(51) Int. Cl. A61B 8/00; A61M 25/02

(52) U.S. Cl. CPC A61B 8/4236; A61M 25/02; A61M 2025/0206; A61M 2025/0253

(57) ABSTRACT

A medical device anchor is disclosed. The apparatus consists of an anchor base with an adhesive side that is attached to a location proximate the patient. The second side of the anchor base includes hook and loop type fasteners that cooperate with a medical device retaining strap that also includes hook and loop type fasteners. A cable/sheath anchor base is also provided to secure any cables, sheath, wires, or the like that may extend from the medical device being used. The cable/sheath anchor base is also provided with an adhesive side and a hook and loop fastener side that engages a cable/sheath anchor strap in a similar manner to that of the medical device anchor base.

### BACKGROUND OF THE INVENTION

1. Field of the Invention

The present invention relates generally to medical devices and, more specifically, to a medical device anchor for releasably holding the medical device to a patient and/or patient's drape covering with the present invention comprising a planar flaccid sheet forming a medical device anchor base, a medical device anchor strap for holding a medical device sheath about the medical device and for then securing the medical device to the medical device anchor base, a probe-cable/sheath anchor base attachable to a patient, a patient's drape covering, or to a structure, such as a table or hospital bed, and a probe-cable/sheath anchor strap securable about the probe cable or cables for releasably holding the cable or cables to the probe-cable/sheath anchor base.

2. Description of the Prior Art

There are other devices designed as holders for medical devices. While these holders may be suitable for the purposes for which they where designed, they would not be as suitable for the purposes of the present invention as heretofore described.

It is thus desirable to provide a simple method of releasably securing a medical device to a patient or patient's drape covering within easy reach for repeated use.

It is further desirable to provide a plurality of anchor pads having an adhesive first side, hook and loop fasteners on the second side, and corresponding hook and loop straps securable about the sheathed medical device and the sheath device cables, which are then themselves releasably attachable to a respective probe-cable/sheath anchor pad which is also provided with an adhesive first side and a hook and loop type fastener second side.

### SUMMARY OF THE PRESENT INVENTION

A primary object of the present invention is to provide a means for releasably positioning a medical device onto a patient or patient's drape covering or garment.

Another object of the present invention is to provide medical device anchor comprising a pair of adhesive hook and loop anchor bases releasably securable to a patient and/or patient drape.

Yet another object of the present invention is to provide medical device anchor straps having hook and loop material on each side thereby securable to the anchor bases.

Still yet another object of the present invention is to provide medical device anchor bases comprising pliable sheet material having a first side and a second side with the first or bottom side having a peelable release sheet covering an adhesive layer providing means for attachment to a patient or patient's drape covering and a second or top side having hook and loop material for attachment of a respective anchor strap.

An additional object of the present invention is to provide medical device anchor straps having anchor base mating hook and loop type fastening material thereon.

A further object of the present invention is to provide a medical device anchor strap of sufficient length to encircle a portion of a medical device so that by virtue of the encircling strap the medical device releasably attaches to the hook and loop portion of the medical device anchor base that is, in turn, releasably attached to either the patient or patient's drape.

A yet further object of the present invention is to provide an anchor strap of sufficient length to encompass the medical device cables so that by virtue of the strap the medical device cables releasably attaches to the hook and loop portion of the medical device anchor base, there again releasably attached to either the patient or patient's drape.

A still yet further object of the present invention is to provide strap means for securing a protective sheath over the medical device and medical device cables.

Additional objects of the present invention will appear as the description proceeds.

The present invention overcomes the shortcomings of the prior art by providing a medical device anchor for holding a medical device to a patient and/or patient's drape covering through an adhesive/hook and loop anchor base; a medical device anchor strap for encircling a portion of a provided medical device thereby securing the medical device to the medical device anchor base; a probe-cable/sheath anchor base attachable to a patient, patient's drape covering, or structure, such as a table or bed, and a probe-cable/sheath anchor strap securable about the medical device's probe-cable(s) for releasably holding the medical device probe-cable(s)/sheath to the probe-cable/sheath anchor base

The foregoing and other objects and advantages will appear from the description to follow. In the description reference is made to the accompanying drawing, which forms a part hereof, and in which is shown by way of illustration specific embodiments in which the invention may be practiced. These embodiments will be described in sufficient detail to enable those skilled in the art to practice the invention, and it is to be understood that other embodiments may be utilized and that structural changes may be made without departing from the scope of the invention. In the accompanying drawing, like reference characters designate the same or similar parts throughout the several views.

The following detailed description is, therefore, not to be taken in a limiting sense, and the scope of the present invention is best defined by the appended claims.

### DESCRIPTION OF THE REFERENCED NUMERALS

Turning now descriptively to the drawings, in which similar reference characters denote similar elements throughout the several views, the figures illustrate the use of the present invention. With regard to the reference numerals used, the following numbering is used throughout the various drawing figures.
10 medical device anchor
12 medical device anchor base
14 medical device anchor base first side (adhesive)
16 medical device second side (hook and loop)
18 medical device anchor strap
20 medical device anchor strap first side (hook and loop)
22 medical device second side (hook and loop)
24 medical device anchor strap end retainer
26 medical device cable/sheath anchor base
28 medical device cable/sheath anchor base first side (adhesive)
30 medical device cable/sheath anchor base second side (hook and loop)
32 medical device cable/sheath anchor strap
34 medical device cable/sheath anchor strap first side (hook and loop)
36 medical device cable/sheath anchor strap second side (hook and loop)
38 medical device cable
40 medical device cable protective sleeve
42 patient drape/cover
P ultrasound probe
C peelable adhesive side cover
U patient
A1 directional arrow
A2 directional arrow

### BRIEF DESCRIPTION OF THE DRAWING FIGURES

In order that the invention may be more fully understood, it will now be described, by way of example, with reference to the accompanying drawing in which:

FIG. 1 is an illustrative view of prior art.

FIG. 2 is an illustrative view of the medical device anchor of the present invention.

FIG. 3 is an orthographic view of the medical device anchor of the present invention.

FIG. 4 is a perspective view of the components of the medical device anchor.

FIG. 5 is a top view of the medical device anchor in use for an ultrasound probe.

FIG. 6 is a continued top view of the medical device anchor in use for an ultrasound probe.

FIG. 7 is another continued top view of the medical device anchor in use for an ultrasound probe.

FIG. 8 is an illustrative view of the medical device anchor in use for an ultrasound probe.

### DETAILED DESCRIPTION OF THE PREFERRED EMBODIMENT

The following discussion describes in detail one embodiment of the invention (and several variations of that embodiment). This discussion should not be construed, however, as limiting the invention to those particular embodiments, practitioners skilled in the art will recognize numerous other embodiments as well. For definition of the complete scope of the invention, the reader is directed to appended claims.

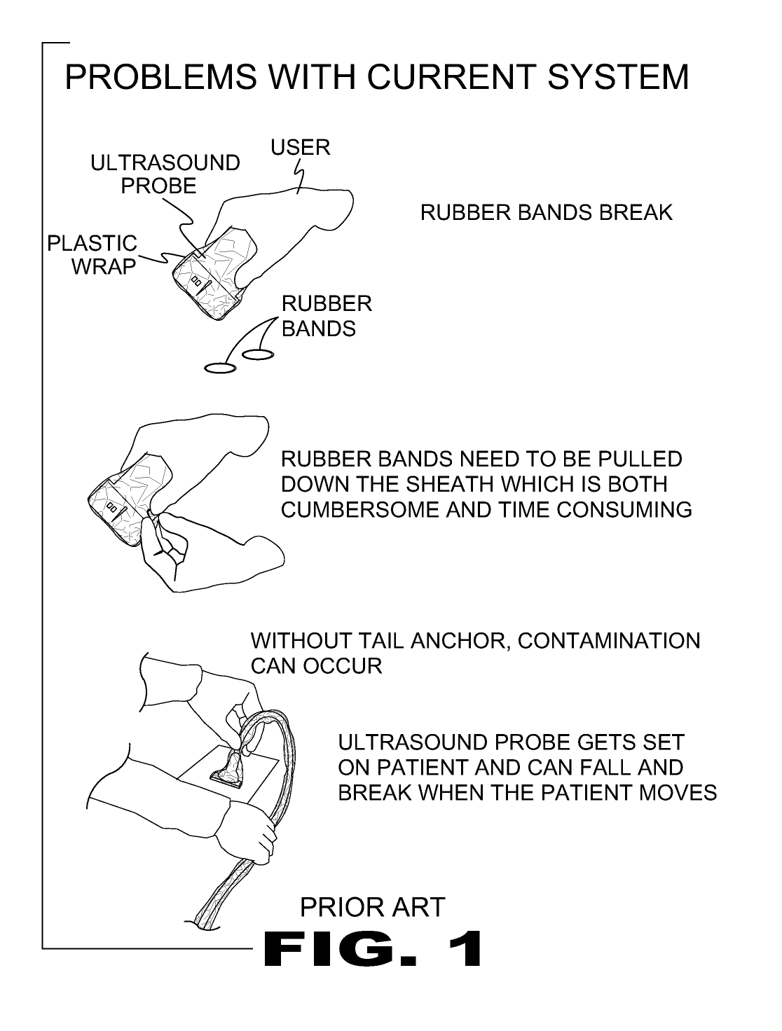

Referring to FIG. 1, shown is an illustrative view of prior art. Medical devices placed on a patient, such as ultrasound probes, are difficult to anchor and tend to fall off sometimes breaking or becoming contaminated. Furthermore, a rubber band is utilized to hold a sheath over the device head but the band often breaks easily while stretching over the device head. Additionally, another band then needs to be stretched over the medical device and pulled down to secure the sheath to the device cables, which is both cumbersome and time consuming. The present invention overcomes this problem by providing a medical device anchor that will releasably hold a medical device and its attendant cables in place.

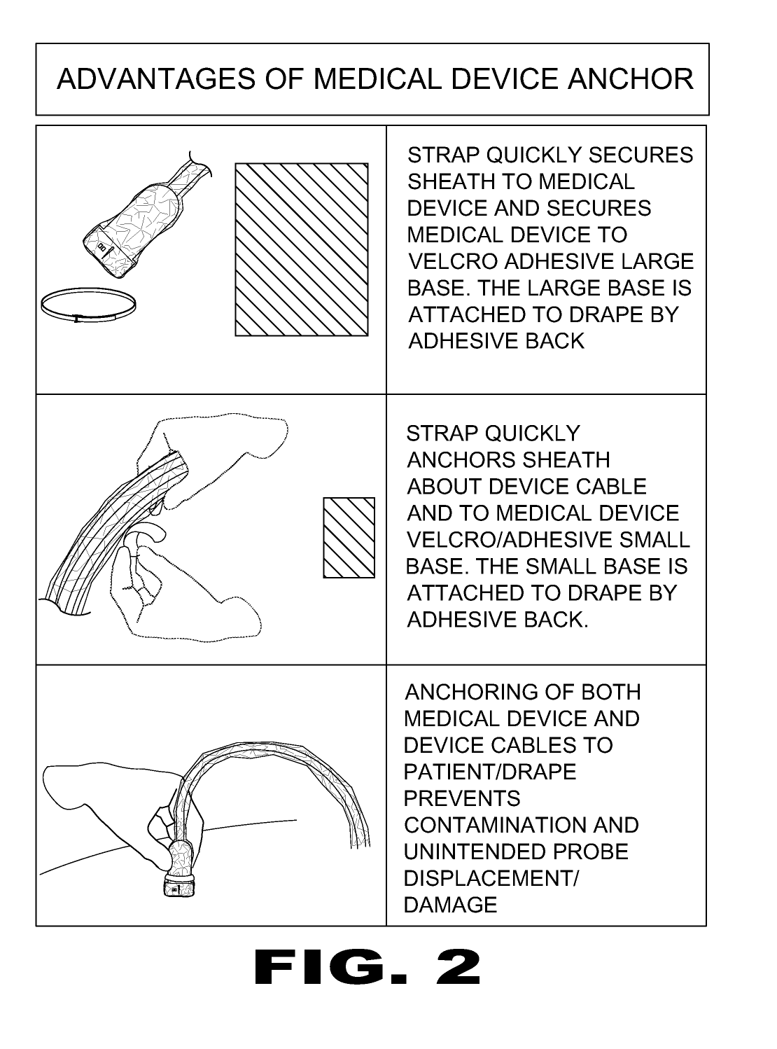

Referring to FIG. 2, shown is an illustrative view of the medical device anchor of the present invention. The present invention provides a means for anchoring a medical device to a patient and/or the patient's drape covering that comprises a medical device anchor base with an adhesive bottom layer and a hook and loop type fastener top layer with a cooperating medical device anchor strap secured about a sheath that covers the medical device wherethen the device and its anchor strap are releasably securable to the anchor base. The cables for the medical device are also sheathed and are further provided with a patient/drape releasable securement that comprises a cable/sheath anchor base and a cable/sheath anchor strap securable to the cable/sheath anchor base.

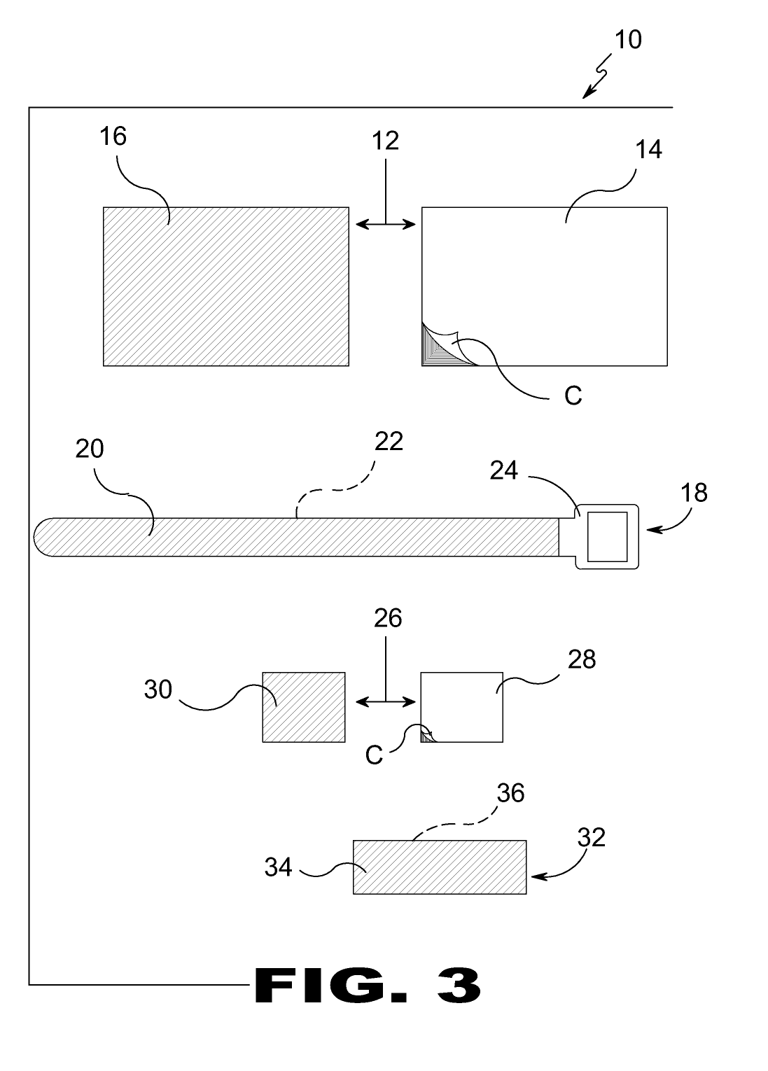

Referring to FIG. 3, shown is an orthographic view of the medical device anchor 10 of the present invention. Shown are the components of the medical device anchor 10 comprising medical device anchor base 12 having a first side (bottom) 14 that includes a peelable adhesive surface area as denoted at C (as will be discussed further below) and also including a second side (top) 16 having one component of a hook and loop type fastening system. Also seen in the Figure is medical device anchor strap 18. Medical device anchor strap 18 has a first side 20 and a second side 22 each of which includes one of either hook or loop type fasteners that cooperate with the corresponding hook or loop type fastener on the second (top) side 16 of the medical device anchor base 12. Medical device anchor strap 18 also includes a strap end retainer 24. Indicated at 26 is the medical device cable sheath anchor base which includes a first side (bottom) 28 having a peelable adhesive surface and a second side (top) 30 having a hook or loop type fastener thereon. Lastly in this Figure is seen the medical device cable sheath anchor strap 32 having a first side 34 and a second side 36, with cooperating hook and loop type fasteners on each.

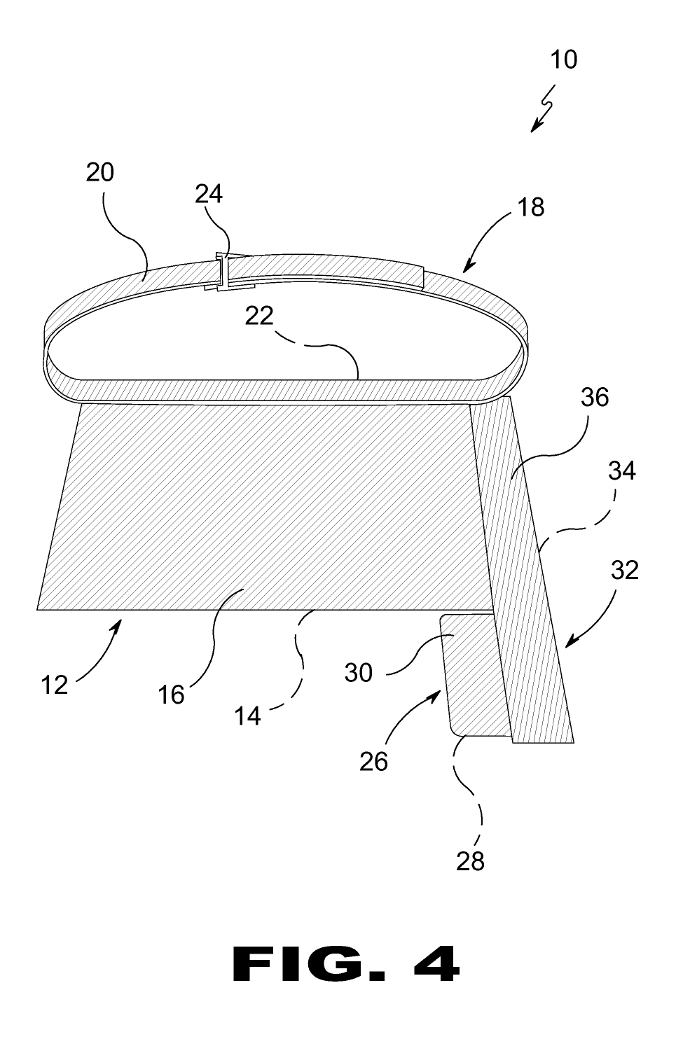

Referring to FIG. 4, shown is a perspective view of the components of the medical device anchor 10. Seen in the Figure is the medical device anchor base 12 with the first (bottom) side 14 that includes the peelable adhesive (discussed below) and the second (top) side 16 having one element of a hook and loop type fastener thereon. The medical device anchor strap 18 is seen with its cooperating first and second sides 20, 22 with their hook and loop fasteners and the strap end retainer 24. The medical device sheath anchor base is indicated at 26 with the first (bottom) side 28 including a peelable adhesive layer and the second (top) side 30 having (as above regarding the anchor base 12) one element of a hook and loop type fastener. The medical device sheath anchor base attaches to the cable sheath anchor strap 32 which includes a first side and a second side 34, 36 both incorporating hook and loop type fasteners as will be discussed below.

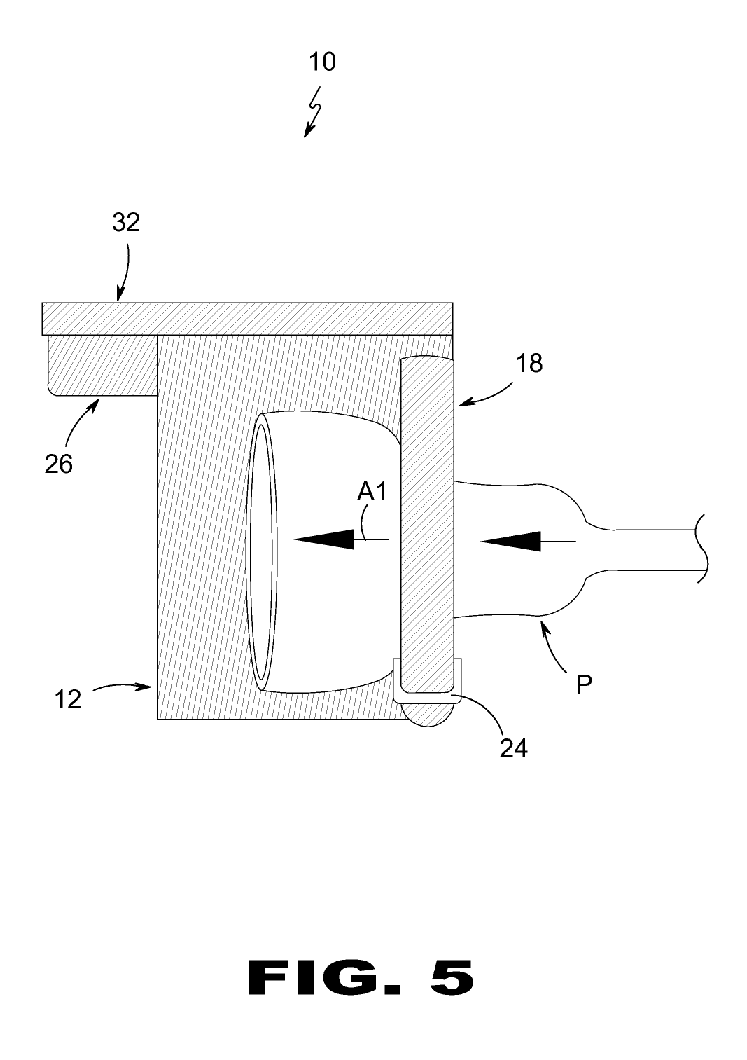

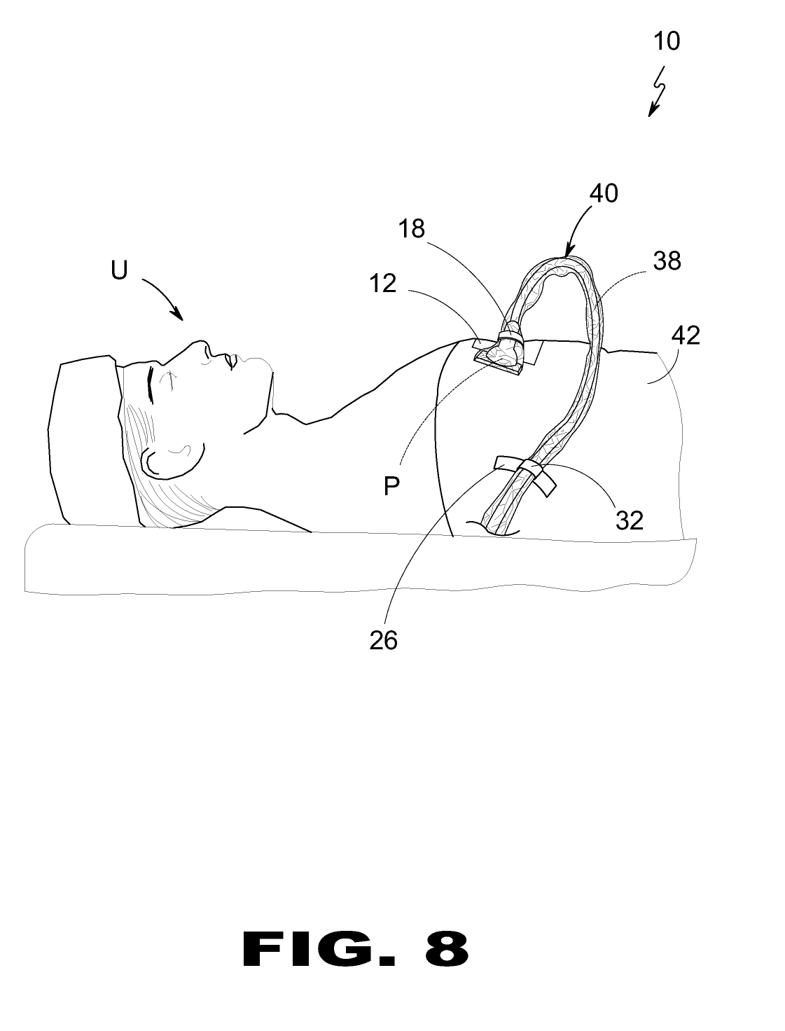

Referring to FIG. 5, shown is a top view of the medical device anchor in use for an ultrasound probe P. Shown, for illustrative purposes, is an ultrasound probe P being inserted into the medical device anchor strap 18 as indicated by directional arrow A1. The anchor strap 18 is held in relation to the medical device anchor base 12 through the cooperating hook and loop type fasteners present on both. In the Figure, a portion of the anchor strap 18 has been placed through the strap end retainer 24. It should be noted that the medical device (in this case the ultrasound probe P) and the medical device anchor strap 18 may be placed in any desired position or at any angle on the medical device anchor base 12 through the use of these hook and loop fasteners as shown. The precise placement of the medical device by means of the present invention is enhanced by the fact that the anchor base 12 itself is positionable proximate the patient by peeling away the adhesive cover C (as seen in FIG. 3) on the first side or bottom 14 of the medical device anchor base 12 and then attaching it as desired to either the drape or cover on the patient or on the patient herself. This will be explained further in the discussion of FIG. 8.

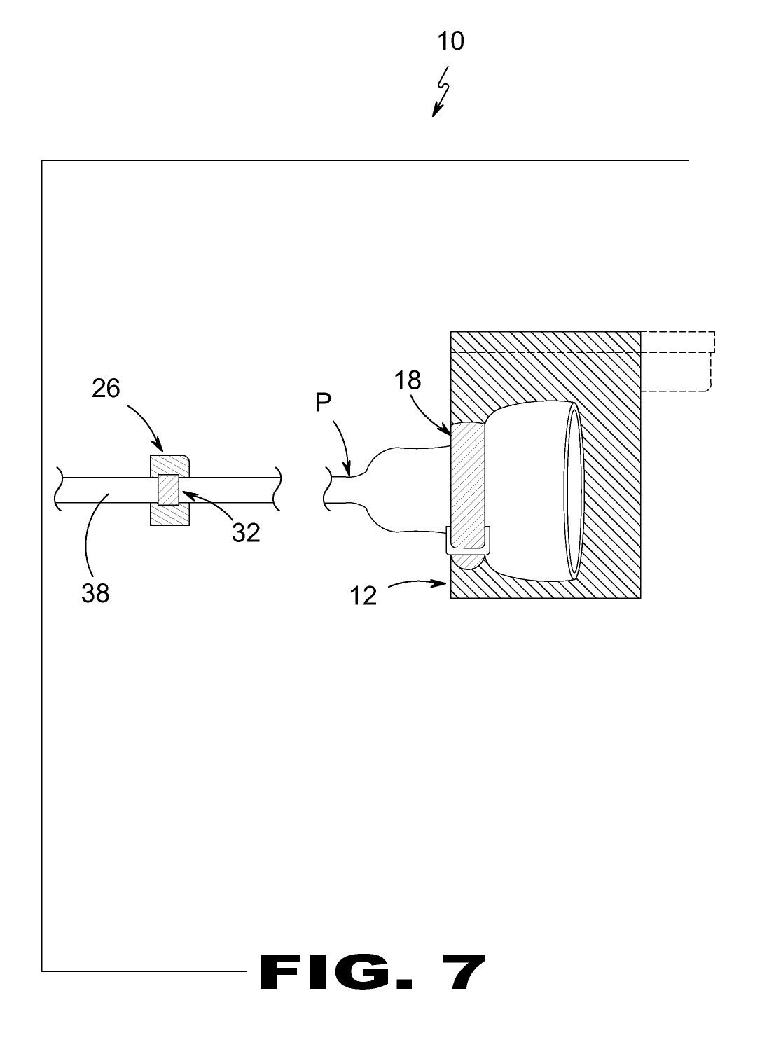

Referring to FIG. 6, shown is a continued top view of the medical device anchor 10 in use with an ultrasound probe. It should be emphasized that though an ultrasound mechanism is seen in the Figures herein, it is only used as a single example of a device that needs to be placed in close proximity or in contact with the patient's body. Likewise, the sheath or protective coverings generally placed around these devices are omitted for clarity in the Figures thus far presented. The device P has been placed within the medical device anchor strap 18 and the strap is cinched in the direction indicated by directional arrow A2. As in the previous Figures, the medical anchor base 12, the cable/sheath anchor base 26, and the cable sheath anchor strap 32 are also shown.

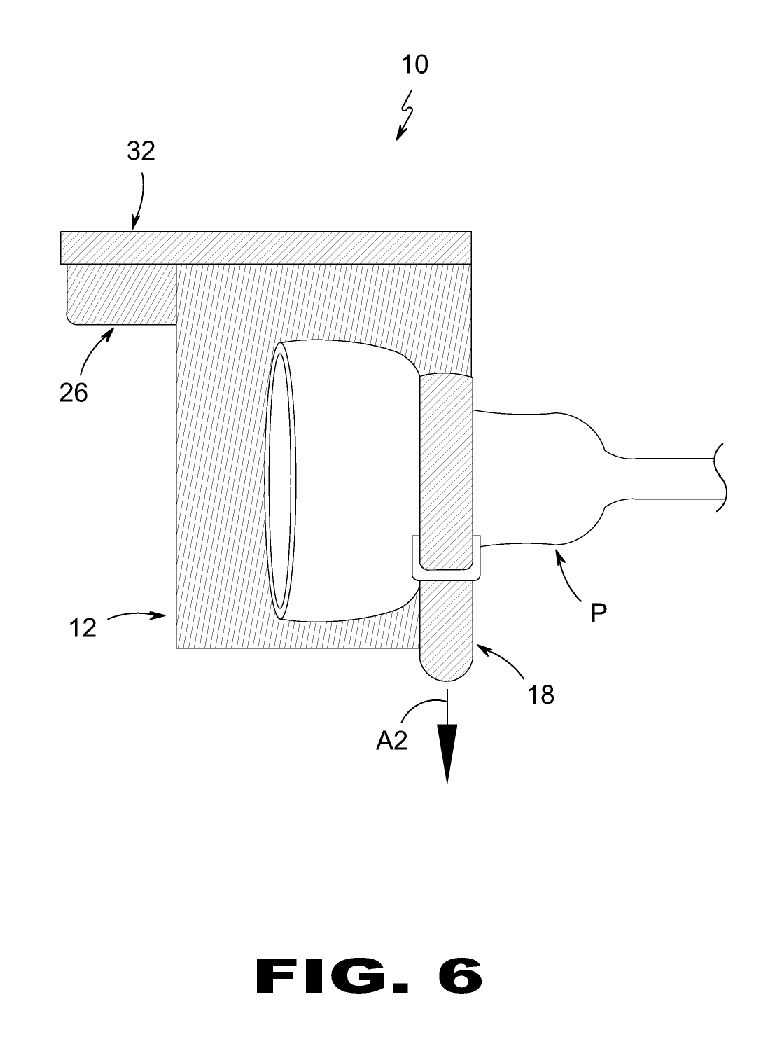

Turning now to FIG. 7, shown is another continued top view of the medical device anchor 10 in use for an ultrasound probe P, as in the previous Figures. Shown, for illustrative purposes, is an ultrasound probe P placed within the medical device anchor strap 18, which is then cinched about the medical device P, as indicated by the directional arrow A2 in FIG. 6, enabling the probe to be attached in the required position on the medical device anchor base 12 that preferably has already been adhesively fixed to a patient's drape once the peelably removable adhesive covering C is removed (as seen in FIG. 3). Also provided is means for selectively positioning and anchoring the medical device cable 38 by placing the cable/sheath anchor base 26 in the desired position and fixing the cable 38 thereto with the cable/sheath anchor strap 32. The cable/sheath anchor base is fixed in position by peeling off the adhesive cover C as seen in FIG. 3 and adhering the adhesive side 28 to the patient, the patient's drape or robe, examination table, bed, or the like to prevent the cable 38 from snagging or getting in the way or contaminating the sterile field. Note that a plurality of cable/sheath anchor bases and cable/sheath anchor straps could be employed if the situation warranted—to string the cable/sheath along a desired path or around various barriers.

Referring now to FIG. 8, shown is an illustrative view of the medical device anchor 10 in use for an ultrasound probe P. Again, the ultrasound probe is used merely as an example of the type of medical device that the current invention could be utilized with. In this Figure the medical device P is enclosed within a sheath or protective sleeve 40. As can be seen from the Figure, the cable/sheath 38 is attached to the drape or cover 42 by both the cable/sheath anchor base 26 and the cable/sheath anchor strap 32. The medical device is positioned through use of the medical device anchor base 12 and the medical device anchor strap 18.

It will be understood that each of the elements described above, or two or more together may also find a useful application in other types of methods differing from the type described above.

While certain novel features of this invention have been shown and described and are pointed out in the annexed claims, it is not intended to be limited to the details above, since it will be understood that various omissions, modifications, substitutions and changes in the forms and details of the device illustrated and in its operation can be made by those skilled in the art without departing in any way from the spirit of the present invention.

What is claimed is new and desired to be protected by Letters Patent is set forth in the appended claims:

1. A medical device anchor for fixing a medical device proximate a patient during a procedure comprising:
a medical device anchor base having a first anchor side and a second anchor side, said first anchor side including an adhesive surface and said second anchor side including hook and loop type fasteners;
a medical device anchor strap having a first strap side and a second strap side, both said first and said second strap sides having cooperating hook and loop type fasteners, whereby;
said first anchor side of said medical device anchor base is adhesively fixed proximate the patient, said medical device anchor strap is secured about the medical device and where said medical device anchor strap and secured medical device are fixed in relation to the patient by said hook and loop type fasteners present on both said medical device anchor base second side and said medical device anchor strap first and second sides.

2. The medical device anchor as claimed in claim 1 where said first anchor side of said medical device anchor base includes a peelable cover to expose said adhesive side.

3. The medical device anchor as claimed in claim 2 where said medical device anchor strap first side comprises hook type fasteners and said medical device anchor strap second side comprises loop type fasteners.

4. The medical device anchor as claimed in claim 3 where said medical device anchor strap includes a strap end retainer.

5. The medical device anchor as claimed in claim 1 where the medical device includes a cable/sheath extending therefrom and where said medical device anchor further includes a cable/sheath anchor base having a first cable/sheath anchor side and a second cable/sheath anchor side, where said first cable/sheath anchor side includes an adhesive surface and said second anchor side includes hook and loop type fasteners, and where said medical device anchor further includes;
a cable/sheath anchor strap having a first cable/sheath strap side and a second cable/sheath strap side, both said first and said second cable/sheath strap sides having cooperating hook and loop type fasteners, whereby;
first cable/sheath anchor side is adhesively fixed in position proximate the patient and where said cable/sheath anchor strap is secured about the cable/sheath extending from the medical device and where said cable/sheath anchor strap and the secured cable/sheath are fixed in relation to the patient by said hook and loop type fasteners present on both said second cable/sheath anchor side and said cable/sheath anchor strap first and second sides.

6. The medical device anchor as claimed in claim 5 where said first anchor side of said medical device anchor base includes a peelable cover to expose said first anchor side adhesive side and where said first cable/sheath anchor side includes a peelable cover to expose said first cable/sheath adhesive side.

7. The medical device anchor as claimed in claim 6 where said medical device anchor strap first side comprises hook type fasteners and said medical device anchor strap second side comprises loop type fasteners and further where said and further where said first cable/sheath strap side comprises hook type fasteners and where said second cable/sheath strap side comprises loop type fasteners.

8. The medical device anchor as claimed in claim 7 where said medical device anchor strap includes a strap end retainer.

9. A method for temporarily anchoring a medical device in a fixed relationship with a patient comprising the steps of:
providing a medical device anchor base where said medical device anchor base includes a first adhesive side and a second device placement side, said device placement side including hook and loop type fasteners;
providing a medical device anchor strap having a first side and a second side, where said both first and second medical device anchor strap sides include cooperating hook and loop type fasteners;
adhesively fixing said medical device anchor base on a desired location proximate the patient;
securing the medical device within said medical device anchor strap using said cooperating hook and loop type fasteners; and
placing the medical device secured within said medical device anchor strap on a desired location on said second device placement side of said medical device anchor base.

10. The method according to claim 9 wherein said first adhesive side of said medical device anchor base includes a peelable cover layer.

11. The method according to claim 10 where said medical device anchor strap further includes a strap retaining end.

12. The method according to claim 9 wherein the medical device includes a cable/sheath extending therefrom further including the step of:
providing a cable/sheath anchor base having a first adhesive side and a second cable/sheath placement side, said cable/sheath placement side including hook and loop type fasteners;
providing a cable/sheath anchor strap having a first side and a second side, where said both first and second cable/sheath anchor strap sides include cooperating hook and loop type fasteners; whereby the cable/sheath extending from the medical device is placed in a fixed relationship with the patient by;
adhesively fixing said cable/sheath anchor base on a desired location proximate the patient;
securing the cable/sheath within said cable/sheath anchor strap using said cooperating hook and loop type fasteners located on said first and second cable/sheath anchor strap sides; and
placing the cable/sheath secured within said cable/sheath anchor strap on a desired location on said second cable/sheath placement side of said cable/sheath anchor base.

13. The method according to claim 12 wherein said first adhesive side of said cable/sheath anchor base includes a peelable cover layer.

14. The method according to claim 13 where said cable/sheath anchor strap further includes a strap retaining end.
